(19) United States
(12) Patent Application Publication
(10) Pub. No.: US 2026/0061258 A1
(43) Pub. Date: Mar. 5, 2026

(54) CONTROL METHOD FOR MOTION EVALUATION SYSTEM, CONTROL METHOD FOR COMPUTER, NON-TRANSITORY COMPUTER-READABLE STORAGE MEDIUM STORING PROGRAM, AND MOTION EVALUATION SYSTEM

(71) Applicant: SEIKO EPSON CORPORATION, Tokyo (JP)

(72) Inventors: Fumikazu SANO, MATSUMOTO-SHI (JP); Shuji UCHIDA, SHIOJIRI-SHI (JP)

(73) Assignee: SEIKO EPSON CORPORATION, Tokyo (JP)

(21) Appl. No.: 19/312,321

(22) Filed: Aug. 28, 2025

(30) Foreign Application Priority Data
Aug. 30, 2024 (JP) 2024-150062

(51) Int. Cl. A63B 24/00; G16H 15/00

(52) U.S. Cl. CPC A63B 24/0062; G16H 15/00; A63B 2024/0071; A63B 2220/16; A63B 2220/31; A63B 2220/53; A63B 2220/836

(57) ABSTRACT

A control method for a motion evaluation system including a sensor attached at a predetermined wearing position below a knee of a subject, and a server device that generates an evaluation report based on a detection result of the sensor, the method includes a first step of causing the sensor to detect motion information indicating a motion below the knee of the subject, a second step of causing the sensor to transmit the motion information to the server device, a third step of causing the server device to acquire length information based on a height of the subject, a fourth step of causing the server device to estimate a movement of a foot of the subject from the received motion information and the acquired length information, and a fifth step of causing the server device to perform an evaluation on the estimated movement of the foot.

The present application is based on, and claims priority from JP Application Serial Number 2024-150062, filed Aug. 30, 2024, the disclosure of which is hereby incorporated by reference herein in its entirety.

### BACKGROUND

### 1. Technical Field

The present disclosure relates to a control method for a motion evaluation system, a control method for a computer, a non-transitory computer-readable storage medium storing a program, and a motion evaluation system.

### 2. Related Art

In a motion performance monitoring system in a team competition environment described in JP-A-2017-74382, a kick of a player as a subject of an evaluation is evaluated using a foot-worn sensor worn on a foot of the player (see JP-A-2017-74382).

JP-A-2017-74382 is an example of the related art.

However, in the related art as described above, when it is difficult to wear the sensor on the foot of the subject, it may be impossible to evaluate the motion of the subject.

### SUMMARY

A control method for a motion evaluation system according to an aspect of the present disclosure is a control method for a motion evaluation system including a sensor attached at a predetermined wearing position below a knee of a subject, and a server device that generates an evaluation report based on a detection result of the sensor, and the method includes a first step of causing the sensor to detect motion information indicating a motion below the knee of the subject, a second step of causing the sensor to transmit the motion information to the server device, a third step of causing the server device to acquire length information based on a height of the subject, a fourth step of causing the server device to estimate a movement of a foot of the subject from the received motion information and the acquired length information, and a fifth step of causing the server device to perform an evaluation on the estimated movement of the foot.

A control method for a motion evaluation system according to an aspect of the present disclosure that uses a sensor attached at a predetermined wearing position below a knee of a subject and a server device to generate an evaluation report based on a detection result of the sensor, the method includes detecting motion information indicating a motion below the knee of the subject, acquiring length information based on a height of the subject, estimating a movement of a foot of the subject from the detected motion information and the acquired length information, and performing an evaluation on the estimated movement of the foot.

A control method for a computer according to an aspect of the present disclosure includes causing the computer to acquire motion information indicating a motion of below a knee of a subject from a sensor attached at a predetermined wearing position below the knee of the subject, causing the computer to acquire length information based on a height of the subject, causing the computer to estimate a movement of a foot of the subject from the acquired motion information and the acquired length information, and causing the computer to perform an evaluation of the estimated movement on the foot.

A non-transitory computer-readable storage medium storing a program according to an aspect of the present disclosure, the program causes a computer to execute the respective steps.

A motion evaluation system according to an aspect of the present disclosure includes a sensor attached at a predetermined wearing position below a knee of a subject and detecting motion information indicating a motion below the knee of the subject, and a server device receiving the motion information from the sensor, estimating a movement of a foot of the subject from the received motion information and length information based on a height of the subject, and performing an evaluation on the estimated movement of the foot.

### BRIEF DESCRIPTION OF THE DRAWINGS

FIG. 1 shows a configuration example of a motion evaluation system according to an embodiment.

FIG. 2 shows a configuration example of a server device according to the embodiment.

FIG. 3A shows a definition of a below-knee part according to the embodiment.

FIG. 3B shows a below-knee length according to the embodiment.

FIG. 3C shows an example of a first wearing position of a measurement device according to the embodiment.

FIG. 3D shows an example of a second wearing position of the e measurement device according to the embodiment.

FIG. 4 is a graph showing an example of a relationship between a height and the below-knee length according to the embodiment.

FIG. 5 shows a definition of an inclination angle of a foot according to the embodiment.

FIG. 6 shows an example of measurement data including kick data according to the embodiment.

FIG. 7 is a graph showing an example of a relationship between an elapsed time and a foot speed according to the embodiment.

FIG. 8A shows an example of changes in posture of a human during a kick according to the embodiment.

FIG. 8B is a graph showing an example of a relationship between a foot speed and a knee speed with respect to an elapsed time in a kick having a good image according to the embodiment.

FIG. 8C is a graph showing an example of a relationship between the foot speed and the knee speed with respect to the elapsed time in a kick having a poor image according to the embodiment.

FIG. 9A shows an example of a movement of a foot of a human during a kick according to the embodiment.

FIG. 9B is a graph showing an example of a relationship between an elapsed time and a foot angle according to the embodiment.

FIG. 10 shows an example of a procedure of processing performed by the server device according to the embodiment.

### DESCRIPTION OF EMBODIMENTS

An embodiment will hereinafter be described with reference to the drawings.

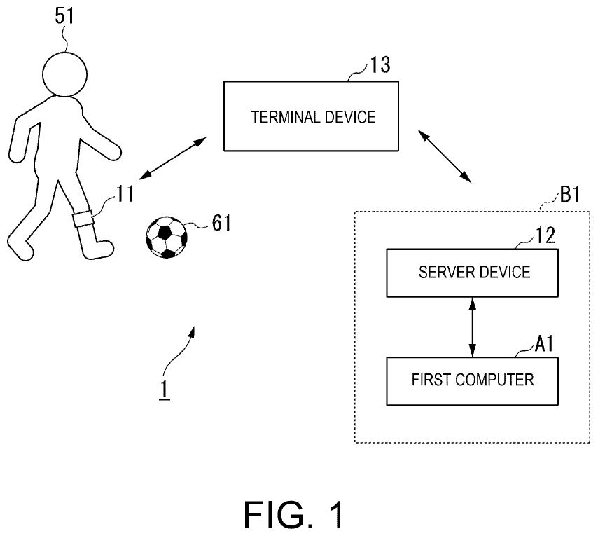

FIG. 1 shows a configuration example of a motion evaluation system 1 according to the embodiment.

The motion evaluation system 1 includes a measurement device 11, a server device 12, a terminal device 13, and a first computer A1.

Further, FIG. 1 illustrates a user 51 and a ball 61 kicked by the user 51.

In the example of FIG. 1, a management section B1 including the server device 12 and the first computer A1 is illustrated.

Here, in the present embodiment, a case where the measurement device 11 and the server device 12 communicate with each other via the terminal device 13 is described, but, for example, when the measurement device 11 and the server device 12 communicate with each other without using the terminal device 13, the terminal device 13 is not necessarily provided in the motion evaluation system 1.

Although not illustrated in the example of FIG. 1, for example, the devices may communicate with each other via a base station device, a relay device, or the like (not illustrated).

In the present embodiment, for example, wireless communication is used as the communication, but there may be a portion where wired communication is used.

In the present embodiment, the first computer A1 that can communicate with the server device 12 is provided, but the first computer A1 is not necessarily provided.

In the example of FIG. 1, when the first computer A1 is not provided in the motion evaluation system 1, the management section B1 is substantially equal to the server device 12.

The function of the first computer A1 may be provided in the server device 12.

The user 51 is a human, and is a child who plays soccer in the present embodiment. In the present embodiment, the ball 61 is a soccer ball.

The measurement device 11 is worn at a predetermined wearing position below the knee of one leg of the user 51. The measurement device 11 may be worn at the wearing position of the user 51 using, for example, a band provided in the measurement device 11 or a band separate from the measurement device 11.

The user 51 may be referred to as, for example, a subject or a player.

The measurement device 11 includes one or more sensors that detect a predetermined physical quantity.

The measurement device 11 may include, for example, an IMU sensor including an inertial measurement unit (IMU). The IMU sensor may measure, for example, an acceleration and an angular velocity.

The measurement device 11 and the terminal device 13 perform wireless communication.

In the present embodiment, the wireless communication may be Bluetooth (registered trademark) Low Energy wireless communication.

Here, the measurement device 11 may include any sensor, and may include, for example, two or more sensors.

The measurement device may be referred to as, for example, a measurement instrument. For example, when the measurement device is equivalent to one sensor, the measurement device may be referred to as a sensor. For example, when the measurement device includes two or more sensors, the measurement device may be referred to as a sensor unit or the like.

The measurement may be referred to as, for example, metering, detection, or sensing.

The value measured by the measurement device 11 may be referred to as, for example, a measurement value, a detection value, or an actual measurement value.

The terminal device 13 may be, for example, a smartphone, a tablet terminal, or a laptop computer.

The terminal device 13 and the server device 12 communicate with each other via a network such as the Internet.

Here, as an example, the terminal device 13 may be held by a coach, a manager, a parent, or the like of the user 51.

The first computer A1 may be operated by a predetermined operator or the like.

The first computer A1 transmits, for example, user information as information on the user 51 to the server device 12.

The first computer A1 and the server device 12 communicate with each other in a wired or wireless manner.

The user 51, the person who operates the terminal device 13, and the person who operates the first computer A1 may be, for example, all different persons, some of them may be the same person, or all of them may be the same person.

The server device 12 performs a predetermined evaluation on the motion of the user 51.

In the present embodiment, schematically, the measurement device 11 transmits measurement data to the terminal device 13, and the terminal device 13 transmits the measurement data to the server device 12.

The server device 12 performs a predetermined evaluation on the motion of the user 51 based on the user information received from the first computer A1 and the measurement data received from the terminal device 13.

The server device 12 transmits the user information and an evaluation result to the terminal device 13.

The terminal device 13 displays the evaluation result received from the server device 12 on a screen. The terminal device 13 may also display the user information received from the server device 12 on the screen.

The terminal device 13 may control the measurement performed by the measurement device 11.

Here, in the present embodiment, for convenience of description, the one user 51 is described as an example. However, for example, the motion evaluation system 1 according to the present embodiment may be applied to a plurality of users, and in this case, the same processing as that in the present embodiment is performed for each user. Further, in this case, information on each of the plurality of users may be, for example, stored as user information.

For example, when the motion evaluation system 1 is applied to a soccer class, the plurality of users may be children who take the soccer class.

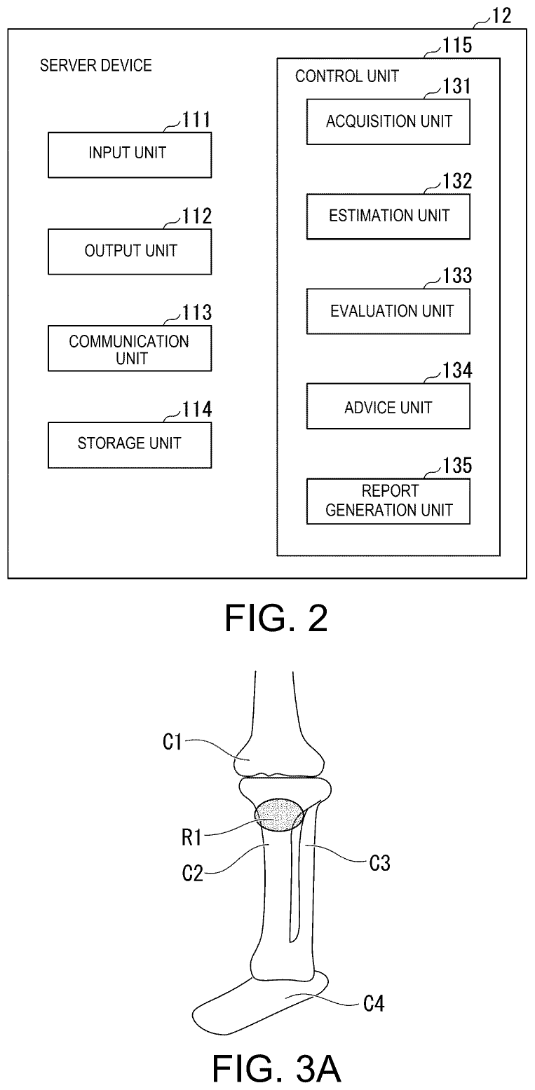

FIG. 2 shows a configuration example of the server device 12 according to the embodiment.

In the present embodiment, the server device 12 is configured using a computer.

The server device 12 includes an input unit 111, an output unit 112, a communication unit 113, a storage unit 114, and a control unit 115.

The control unit 115 includes an acquisition unit 131, an estimation unit 132, an evaluation unit 133, an advice unit 134, and a report generation unit 135.

The input unit 111 may have, for example, a function of inputting information output from an external device (not illustrated).

When the function of the first computer A1 is incorporated in the server device 12, the input unit 111 may have, for example, a function of inputting an instruction or the like based on an operation performed by an operator or the like (not illustrated).

The output unit 112 may have, for example, a function of outputting information to an external device (not illustrated).

When the function of the first computer A1 is incorporated in the server device 12, the output unit 112 may have, for example, a function of displaying and outputting information to be displayed on a display screen (not illustrated), a function of outputting sound information to a speaker (not illustrated), or the like.

The communication unit 113 has a function of performing communication.

In the present embodiment, the communication unit 113 communicates with the terminal device 13 via a network such as the Internet.

Note that the communication unit 113 is illustrated separately from the input unit 111 and the output unit 112 in the present embodiment, but, for example, a function of a reception unit among the functions of the communication unit 113 may be provided to the function of the input unit 111, and a function of a transmission unit among the functions of the communication unit 113 may be provided to the function of the output unit 112.

The storage unit 114 stores information.

Note that the server device 12 may be configured to use an external database (not illustrated) instead of the storage unit 114 or together with the storage unit 114 to perform at least one of storing information in that database and acquiring information from that database.

The control unit 115 performs various kinds of processing or control in the server device 12.

In the present embodiment, the control unit 115 includes a predetermined processor such as a CPU (Central Processing Unit), and performs various kinds of processing or control by the processor executing a control program.

For example, the control program may be stored in the storage unit 114.

The acquisition unit 131 acquires data necessary for processing.

For example, the acquisition unit 131 acquires data stored in the storage unit 114, data received by the communication unit 113, or data input by the input unit 111.

The data may be called information or the like instead.

The estimation unit 132 performs a predetermined estimation. In the present embodiment, for example, the estimation unit 132 may perform an estimation of a below-knee length, an estimation of a foot speed, and an estimation of a speed of the ball 61.

The evaluation unit 133 performs a predetermined evaluation. In the present embodiment, for example, the evaluation unit 133 may perform an evaluation on a kick when the user 51 kicks the ball 61.

The advice unit 134 performs processing on predetermined advice. In the present embodiment, the advice is advice on an evaluation result.

The report generation unit 135 generates a predetermined report. In the present embodiment, the report is a report on an evaluation result, and may include advice information.

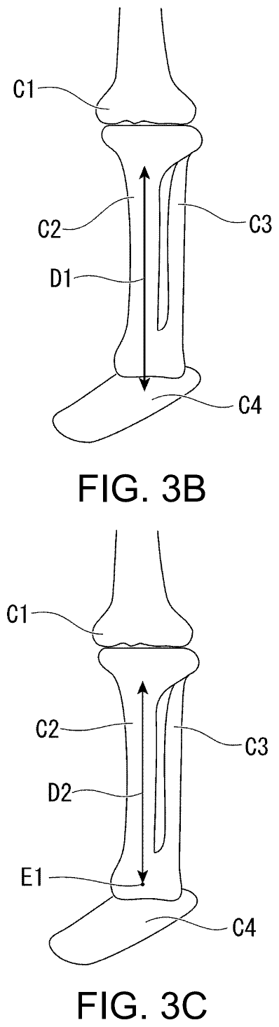

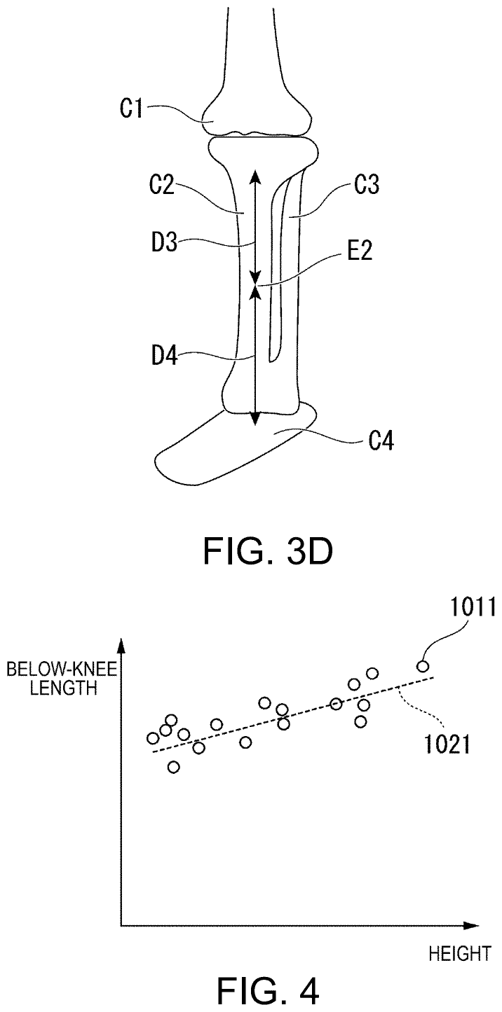

In the examples of FIGS. 3A to 3D, for convenience of description, the position closer to the foot with respect to the position of the human knee is referred to as a lower side, and the position closer to the knee with respect to the position of the foot is referred to as an upper side.

FIG. 3A shows a definition of a below-knee part according to the embodiment.

FIG. 3A shows a patella C1, a tibia C2 and a fibula C3 below the patella C1, and a tarsal C4 below the tibia C2 and the fibula C3 as a schematic bone structure in the vicinity of a human foot.

In the present embodiment, a below-knee region immediately below the knee is a region of the tibia C2 below the patella C1 and not over the patella C1.

In the example of FIG. 3A, a first region R1 as an example of the below-knee region is schematically illustrated. In the present embodiment, a position of one point within the first region R1 is used as a below-knee position.

In the present embodiment, as an example, the measurement device 11 is worn at the wearing position as the below-knee position.

FIG. 3B shows a length from a below-knee part to the foot according to the embodiment.

Similarly to FIG. 3A, FIG. 3B illustrates the patella C1, the tibia C2 and the fibula C3 below the patella C1, and the tarsal C4 below the tibia C2 and the fibula C3.

FIG. 3B shows a first length D1 corresponding to the length from the below-knee part to the foot in the present embodiment.

Here, in the present embodiment, the length from the below-knee part to the tarsal C4 is used as the length from the below-knee part to the foot.

In the present embodiment, the length from the below-knee part to the foot is also referred to as a below-knee length.

FIG. 3C shows an example of a first wearing position E1 of the measurement device 11 according to the embodiment.

Similarly to FIG. 3A, FIG. 3C illustrates the patella C1, the tibia C2 and the fibula C3 below the patella C1, and the tarsal C4 below the tibia C2 and the fibula C3.

In the example of FIG. 3C, the first wearing position E1 as a position above the foot is shown as another example of the position where the measurement device 11 is worn.

In the example of FIG. 3C, a second length D2 corresponding to the length from the below-knee part to the foot is shown.

Here, for example, the position above the foot is at the lower end of the tibia C2.

In the example of FIG. 3C, the measurement device 11 is worn above the foot and measures a foot speed. The speed of the first wearing position E1, which is a predetermined position below the knee, may be estimated from the foot speed and the length from the below-knee part to the foot.

FIG. 3D shows an example of a second wearing position E2 of the measurement device 11 according to the embodiment.

Similarly to FIG. 3A, FIG. 3D illustrates the patella C1, the tibia C2 and the fibula C3 below the patella C1, and the tarsal C4 below the tibia C2 and the fibula C3.

In the example of FIG. 3D, the second wearing position E2, which is the center position of the tibia C2 in the longitudinal direction, is shown as another example of the position where the measurement device 11 is worn.

Further, in the example of FIG. 3D, a third length D3 as a length from the second wearing position E2 to the below-knee part and a fourth length D4 as a length from the second wearing position E2 to the tarsal C4 are shown.

Here, each of the third length D3 and the fourth length D4 may be regarded as, for example, a half of the length from the below-knee part to the foot.

In the example of FIG. 3D, the measurement device 11 is worn at the center position of the tibia C2 in the longitudinal direction, and measures the speed at the position. The speed of the below-knee part may be estimated from the speed of the position and the third length D3. The foot speed may be estimated from the speed of the position and the fourth length D4.

FIG. 4 is a graph illustrating an example of a relationship between a height and the below-knee length according to the embodiment.

In the graph, the horizontal axis represents the height [cm] of a human, and the vertical axis represents the below-knee length [cm] of the human.

In the graph, a plurality of circles shows a plurality of data points as results of actual measurement. In the example of FIG. 4, only one data point is denoted by a sign 1011 for simplicity of the illustration.

Further, the graph shows a first characteristic line 1021 which is a straight line based on a model formula obtained based on the plurality of data points.

In the present embodiment, a below-knee length can be estimated from a height based on the first characteristic line 1021.

Here, the model formula is a model formula for estimating a below-knee length from a height.

For example, the model formula may be acquired by collecting data representing correspondences between the height and the below-knee length and performing fitting using the least squares method or the like based on the data.

As an example, the model formula may be {(below-knee length)=(first value)×(height)+(second value)}, and (first value) and (second value) may be obtained by linear approximation.

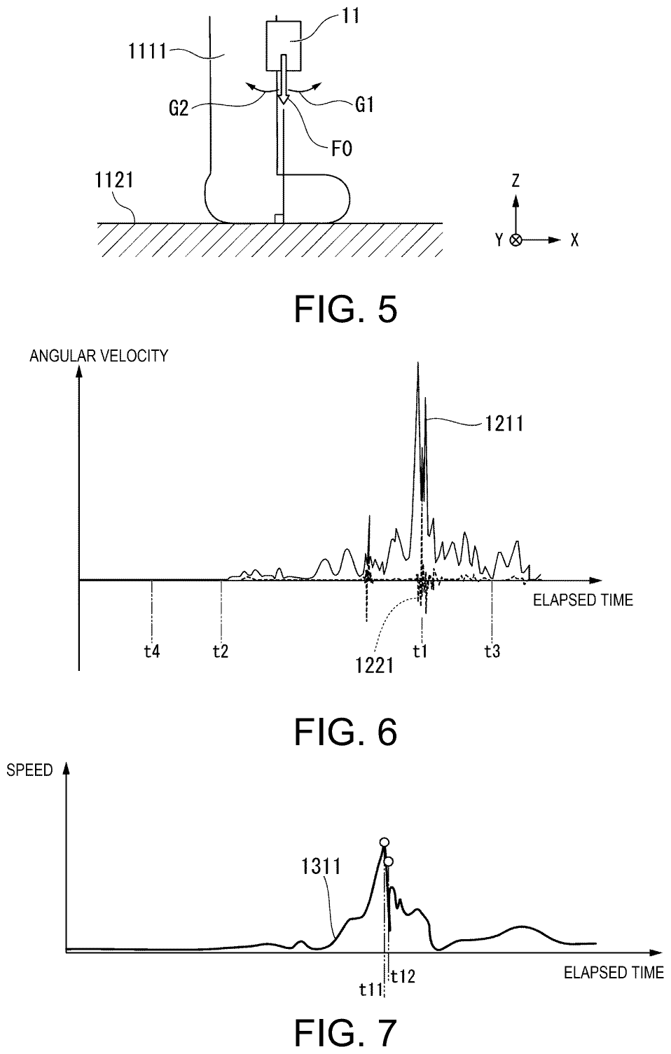

FIG. 5 shows a definition of an inclination angle of the foot according to the embodiment.

FIG. 5 illustrates XYZ orthogonal coordinate axes, which are three-dimensional orthogonal coordinate axes, for convenience of description.

In the present example, a direction from minus to plus of the X-axis is a frontward direction, and a direction from plus to minus of the X-axis is a rearward direction. Further, in the present example, a direction from minus to plus of the Z-axis is an upward direction, and a direction from plus to minus of the Z-axis is a downward direction in which gravity is applied.

FIG. 5 shows a leg part 1111 including a lower leg and a foot below a human knee. The human stands straight facing in the frontward direction, and the foot is in contact with a ground 1121.

In the example of FIG. 5, the measurement device 11 is worn at a position in the frontward direction of the below-knee part. In the present example, it is assumed that the measurement device 11 is fixed at a wearing position in the below-knee part.

In the example of FIG. 5, when the human stands straight facing in the frontward direction, a predetermined direction F0 vertically downward from a predetermined location of the measurement device 11 is set as a reference direction. That is, a state in which the predetermined direction F0 viewed from the measurement device 11 forms an angle of 90 degrees with respect to the ground 1121 is set as a reference.

When the human raises the foot in the frontward direction, the predetermined direction F0 is inclined in a direction of a first inclination G1, and in the present embodiment, the inclination is regarded as an inclination toward the plus side with respect to 90 degrees.

Conversely, when the human raises the foot in the rearward direction, the predetermined direction F0 is inclined in a direction of a second inclination G2, and in the present embodiment, the inclination is regarded as an inclination toward the minus side with respect to 90 degrees.

FIG. 6 shows an example of measurement data including kick data according to the embodiment.

In a graph shown in FIG. 6, the horizontal axis represents an elapsed time [second], and the vertical axis represents an angular velocity [degree per second].

FIG. 6 shows a second characteristic 1211 representing an angular velocity norm obtained based on the and a third characteristic 1221 measurement data representing a difference in angular velocity norms.

FIG. 6 shows a first time t1 to a fourth time t4 for the elapsed time on the horizontal axis.

The first time t1 represents a time when the difference in angular velocity norm is the largest.

The second time t2 represents a time when the angular velocity norm is less than a predetermined value earlier than the first time t1.

The third time t3 represents a time after a lapse of a predetermined time from the first time t1. In the present embodiment, the time is regarded as a time when a kick ends. Any time may be set as the predetermined time.

The fourth time t4 represents a time a predetermined time earlier than the second time t2 before the kick. As the predetermined time, any time may be set, and for example, 1 second may be set.

In the present embodiment, the measurement data between the fourth time t4 and the third time t3 is regarded as data corresponding to one kick.

Thus, data corresponding to the kick can be extracted from the measurement data.

The extraction of data may be referred to as, for example, clipping of data.

FIG. 7 is a graph showing an example of a relationship between an elapsed time and a foot speed according to the embodiment.

In the graph shown in FIG. 7, the horizontal axis represents the elapsed time [second], and the vertical axis represents the foot speed [m/second].

FIG. 7 shows a fourth characteristic 1311 representing the foot speed.

FIG. 7 shows an eleventh time t11 and a twelfth time t12 for the elapsed time on the horizontal axis.

The eleventh time t11 represents a time when the foot speed is the maximum speed.

The twelfth time t12 represents an impact speed. Here, the impact indicates that the foot of the user 51 hits the ball 61. The impact speed represents the foot speed when the foot of the user 51 hits the ball 61.

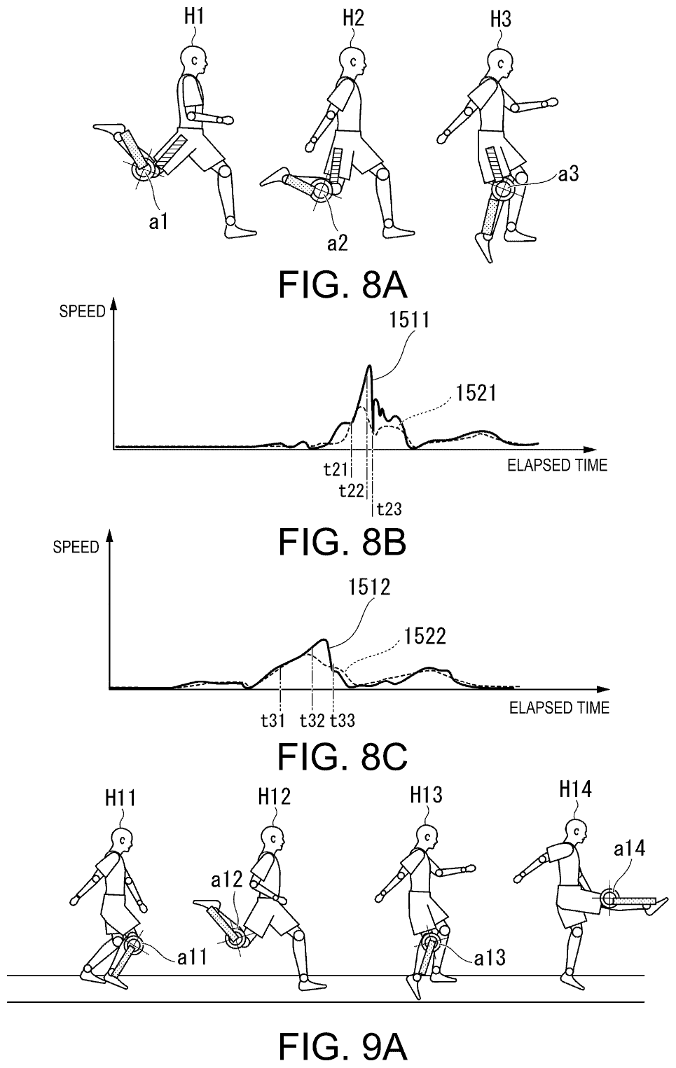

A kick having a good image and a kick having a poor image will be described with reference to FIGS. 8A, 8B, and 8C.

FIG. 8A shows an example of changes in posture of a human during a kick according to the embodiment.

FIG. 8A illustrates a first posture H1 of the user 51, a second posture H2 of the user 51, and a third posture H3 of the user 51 as examples of the posture of the human during the kick.

When the user 51 kicks the ball 61, the posture changes in the order of the first posture H1, the second posture H2, and the third posture H3.

FIG. 8A illustrates a first knee a1 as the knee of the user 51 in the first posture H1, a second knee a2 as the knee of the user 51 in the second posture H2, and a third knee a3 as the knee of the user 51 in the third posture H3.

In the first posture H1, the user 51 swings down the first knee a1, and the speed of the first knee a1 is accelerated.

In the second posture H2, the second knee a2 decelerates, and the user 51 swings down the foot.

In the third posture H3, the foot of the user 51 accelerates and hits the ball 61.

FIG. 8B is a graph showing an example of a relationship between a foot speed and a knee speed with respect to an elapsed time in the kick having the good image according to the embodiment.

In the graph shown in FIG. 8B, the horizontal axis represents the elapsed time [second], and the vertical axis represents the foot speed [m/second].

FIG. 8B shows a fifth characteristic 1511 representing the foot speed and a sixth characteristic 1521 representing the knee speed.

FIG. 8B shows a twenty-first time t21 to a twenty-third time t23 for the elapsed time on the horizontal axis.

The twenty-first time t21 represents a time in the first posture H1 shown in FIG. 8A.

The twenty-second time t22 represents a time in the second posture H2 shown in FIG. 8A.

The twenty-third time t23 represents a time in the third posture H3 illustrated in FIG. 8A.

FIG. 8C is a graph showing an example of a relationship between the foot speed and the knee speed with respect to the elapsed time in the kick having the poor image according to the embodiment.

In the graph shown in FIG. 8C, the horizontal axis represents the elapsed time [second], and the vertical axis represents the foot speed [m/second].

FIG. 8C illustrates a seventh characteristic 1512 representing the foot speed and an eighth characteristic 1522 representing the knee speed.

Further, FIG. 8C shows a thirty-first time t31 to a thirty-third time t33 for the elapsed time on the horizontal axis.

The thirty-first time t31 represents the time in the first posture H1 shown in FIG. 8A.

A thirty-second time t32 represents the time in the second posture H2 illustrated in FIG. 8A.

The thirty-third time t33 represents the time in the third posture H3 shown in FIG. 8A.

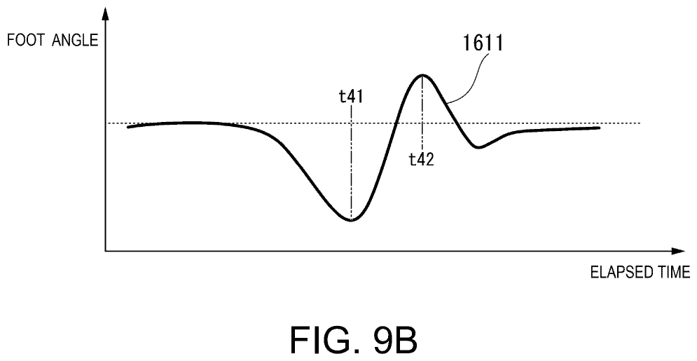

Referring to FIGS. 9A and 9B, a movement of the foot during a kick will be described.

FIG. 9A shows an example of the movement of the human foot during a kick according to the embodiment.

FIG. 9A illustrates an eleventh posture H11 of the user 51, a twelfth posture H12 of the user 51, a thirteenth posture H13 of the user 51, and a fourteenth posture H14 of the user 51 as examples of the movement of the human foot during the kick.

When the user 51 kicks the ball 61, the posture changes in the order of the eleventh posture H11, the twelfth posture H12, the thirteenth posture H13, and the fourteenth posture H14.

FIG. 9A illustrates an eleventh knee a11 as the knee of the user 51 in the eleventh posture H11, a twelfth knee a12 as the knee of the user 51 in the twelfth posture H12, a thirteenth knee a13 as the knee of the user 51 in the thirteenth posture H13, and a fourteenth knee a14 as the knee of the user 51 in the fourteenth posture H14.

In the present example, whether the foot is firmly raised is evaluated based on the twelfth posture H12.

Further, in the present example, whether the foot is firmly swung is evaluated based on the fourteenth posture H14.

FIG. 9B is a graph showing an example of a relationship between an elapsed time and a foot angle according to the embodiment.

In the graph shown in FIG. 9B, the horizontal axis represents the elapsed time [second], and the vertical axis represents the foot angle [degree].

FIG. 9B shows a ninth characteristic 1611 representing the foot angle.

FIG. 9B illustrates a forty-first time t41 and a forty-second time t42 for the elapsed time on the horizontal axis.

The forty-first time t41 represents a time in the twelfth posture H12 illustrated in FIG. 9A.

The forty-second time t42 represents a time in the fourteenth posture H14 illustrated in FIG. 9A.

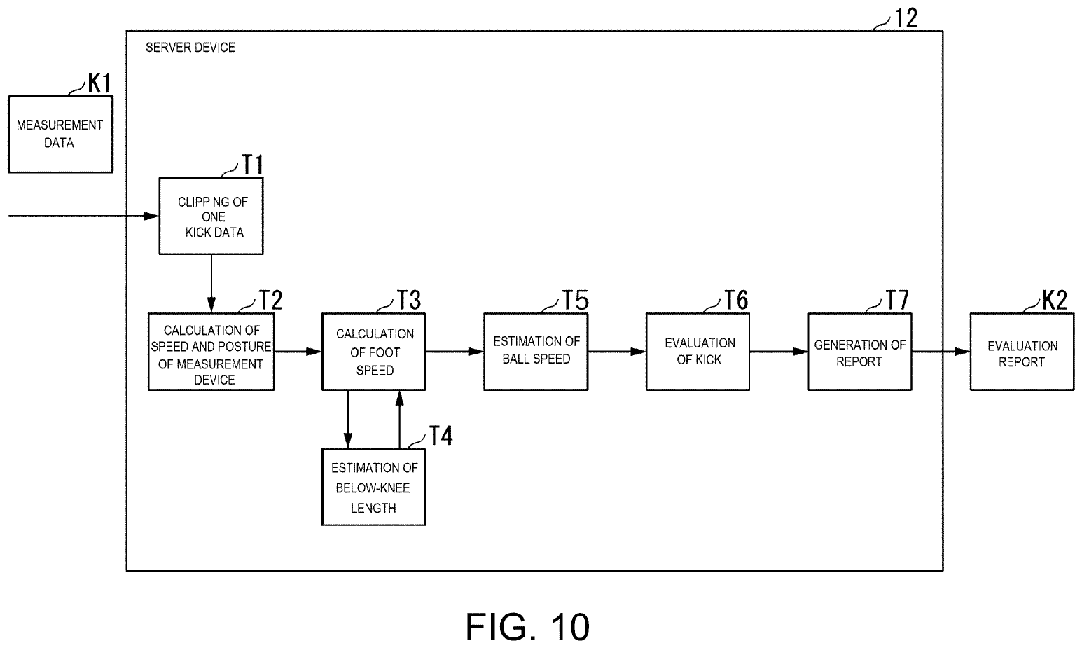

FIG. 10 shows an example of a procedure of processing performed by the server device 12 according to the embodiment.

In processing T1, the acquisition unit 131 acquires measurement data K1, and the estimation unit 132 clips data of one kick from the measurement data K1.

In processing T2, the estimation unit 132 calculates a speed and a posture of the measurement device 11.

In processing T3, the estimation unit 132 calculates a foot speed of the user 51.

Concurrently, in processing T4, the estimation unit 132 estimates a below-knee length of the user 51 based on the height of the user 51. Then, the estimation unit 132 calculates the foot speed of the user 51 using the estimated below-knee length.

As another example, the estimation unit 132 may not perform the processing T4 when the information on the below-knee length of the user 51 is acquired by the acquisition unit 131.

In processing T5, the estimation unit 132 estimates the foot speed of the user 51 and estimates the speed of the ball 61 kicked by the user 51 based on the estimation result.

In processing T6, the evaluation unit 133 evaluates the kick performed by the user 51.

In processing T7, the report generation unit 135 generates a predetermined evaluation report K2.

Concurrently, the advice unit 134 may generate predetermined advice, and the evaluation report K2 may contain the advice.

Here, the flow of the entire processing performed in the motion evaluation system 1 according to the present embodiment will be described more specifically.

In the present embodiment, it is assumed that the measurement device 11 includes an IMU sensor.

In the present embodiment, it is assumed that the measurement device 11 is attached to the below-knee position of the user 51, but as another example, when the measurement device 11 is attached to another part below the knee, the arithmetic expression or the like to be used may be adjusted according to the part.

In the preparation for measurement, information on the user 51 whose kick is evaluated is input from the first computer A1 to the server device 12 using a web browser.

Here, for example, the information on the user 51 includes information such as a name and a date of birth, and includes information on a height in the present embodiment. In the present embodiment, a case where the below-knee length is estimated from the height is shown, but when such an estimation is not performed, the height information may not be input to the server device 12.

The server device 12 stores the input information on the user 51 in the storage unit 114.

The processing for measurement will be described.

The user 51 wears the measurement device 11 at a predetermined part. In the present embodiment, the predetermined part is at the below-knee position.

Then, the measurement device 11 worn by the user 51 starts measurement. Here, the measurement device 11 may start the measurement, for example, in response to reception of a predetermined instruction from the terminal device 13. The terminal device 13 may be operated by the user 51, a coach, or the like to transmit the predetermined instruction to the measurement device 11.

As another example, the measurement device 11 may be directly operated to start the measurement, or the measurement device 11 may be configured to constantly perform the measurement.

At the start of the measurement, for example, the measurement device 11 starts the IMU sensor and measures acceleration data and angular velocity data at predetermined intervals. The predetermined interval may be, for example, 1 millisecond, and in this case, measurement is performed at 1 KHz.

The measurement device 11 stores data of the measurement result in an internal memory (not shown).

Then, the user 51 kicks the ball 61.

Thereafter, the measurement device 11 stops the measurement. Here, the measurement device 11 may end the measurement, for example, in response to reception of a predetermined instruction from the terminal device 13. The terminal device 13 may be operated by the user 51, a coach, or the like to transmit the predetermined instruction to the measurement device 11.

As another example, the measurement device 11 may be directly operated to end the measurement, or the measurement device 11 may be configured to constantly perform the measurement.

When stopping the measurement, the measurement device 11 stops the IMU sensor and ends the storage in the internal memory.

After ending the measurement, the measurement device 11 performs processing of transmitting measurement data K1, which is data of a measurement result, to the server device 12. Here, for example, the measurement device 11 may perform processing of transmitting the measurement data K1 to the server device 12 in response to reception of a predetermined instruction from the terminal device 13. The terminal device 13 may be operated by the user 51, a coach, or the like to transmit the predetermined instruction to the measurement device 11.

In the present embodiment, the measurement device 11 uploads the measurement data K1 to the terminal device 13, and the terminal device 13 uploads the measurement data K1 to the server device 12 by Internet communication or the like.

The server device 12, for example, analyzes the uploaded measurement data K1, and transmits data of the result to the terminal device 13.

The terminal device 13 displays the data of the result on the screen. Accordingly, the user 51, the coach, or the like can view the data of the result.

Here, the data of the result includes, for example, an evaluation result of the kick.

The processing illustrated in FIG. 10 will be described in more detail with respect to kick evaluation processing performed by the server device 12.

In the processing T1 illustrated in FIG. 10, as illustrated in FIG. 6, the server device 12 clips data of one kick as a subject from the received measurement data K1.

The data of one kick is clipped by, for example, the following procedures P1 to P4.

In the procedure P1, a point having the largest difference in norm of the angular velocities of the three axes is determined as an impact point of the kick. That is, when the foot hits the ball 61, the rotation of the foot is decelerated. The rotation of the foot is, for example, rotation around the knee.

In the example of FIG. 6, the first time t1 corresponds to the time of the impact point.

Here, the norm of the angular velocities of the three axes is a value obtained by summing squares of the respective angular velocities with respect to the X-axis, the Y-axis, and the Z-axis and taking a square root of the sum value. The difference in norm is, for example, a difference between a norm at a certain time point and a norm in the past before the predetermined time with respect to the norms acquired from measurement values obtained at predetermined time intervals. The predetermined time may be, for example, 1 [millisecond].

In the procedure P2, the time is traced back from the impact point, the measurement data K1 is checked, and a point at which the norm of the angular velocities of the three axes is less than a predetermined value is determined as a point before the kick. The predetermined value may be, for example, 10 [degrees per second].

In the example of FIG. 6, the second time t2 corresponds to the time of the point before the kick.

In the procedure point P3, a after a predetermined time has elapsed from the impact point is determined as a kick end point. The predetermined time may be, for example, 200 [milliseconds].

In the example of FIG. 6, the third time t3 corresponds to the time of the kick end point.

In the procedure P4, a point a predetermined time earlier than the point before the kick is determined as a preliminary point. The predetermined time may be, for example, 1 [second].

In the example of FIG. 6, the fourth time t4 corresponds to the time of the preliminary point.

Then, a range from the preliminary point to the kick end point is determined as a subject of the kick data.

Here, in the present example, the time from the preliminary point to the point before the kick is regarded as a time before the start of the kick, and may be used as, for example, a stationary section of the user 51 for calibration of the posture calculation.

In the processing T2 illustrated in FIG. 10, the server device 12 calculates the speed and the posture of the measurement device 11.

In the calculation, the speed and the posture are calculated from the acceleration and the angular velocity for the data section as the subject of the kick data.

In the processing T3 and the processing T4 illustrated in FIG. 10, the server device 12 estimates the below-knee length from the height of the user 51 and calculates the foot speed based on the estimation result.

For example, in the configuration having a foot speed calculation function of calculating the foot speed and a below-knee length estimation function of estimating the below-knee length, the foot speed calculation function may input the height information to the below-knee length estimation function, and the below-knee length estimation function may output the information on the below-knee length corresponding to the height to the foot speed calculation function.

For example, the foot speed may be calculated based on the speed and the posture of the below-knee part and the below-knee length.

In the present embodiment, in the calculation of the foot speed, the region from the below-knee part to the foot is defined as a rigid body. That is, when the region from the below-knee part to the foot is defined as a rigid body, the angular velocity is the same and the rotation amount is the same, and thus the speed and the posture of the foot can be calculated by obtaining the acceleration of the foot from the below-knee length.

In the processing T5 illustrated in FIG. 10, the speed of the ball 61 is estimated in the server device 12.

In the present example, the speed of the ball 61 is estimated based on the foot speed at the impact point.

This estimation may be performed using a model formula. For example, the model formula may be a model formula in which the speed of a ball when a human actually kicks the ball is measured by a speed gun or the like, and the relationship between the foot speed at the impact point measured by the measurement device 11 and the speed of the ball is obtained. In the model formula, the speed of the ball 61 is specified from the foot speed.

In the processing T6 illustrated in FIG. 10, an evaluation of the kick is performed in the server device 12 as illustrated in FIGS. 7, 8A to 8C, and 9A and 9B.

In the present example, the evaluation unit 133 evaluates the following first to fourth evaluation items from the speeds and the postures of the foot and the below-knee part.

For example, each evaluation may be performed using a stepwise score. As an example, evaluations by scores at ten steps may be performed with the worst score as 1 point and the best score as 10 points in increments of 1 point.

As an example, kick data of a high-level player in soccer may be collected, and based on the data, the score when data is close to the numerical value of the data of the player may be set to 10 points, and the score may be set to be gradually lower as the data is worse than the numerical value of the data of the player.

In the evaluation of the kick, schematically, a kick force is measured based on the foot speed at the impact and the estimated speed of the ball 61.

Further, in the evaluation of the kick, whether the impact can be made at the point where the foot speed during the kick is maximized.

The first evaluation item is a result of calculation of the deceleration until the impact based on a difference between the maximum foot speed and an impact speed. That is, the evaluation based on the comparison between the maximum foot speed and the impact speed can be made.

Schematically, the evaluation is good when there is no deceleration until the impact, and the evaluation is poor when the deceleration until the impact is large.

For example, the first evaluation item may be based on the information illustrated in FIG. 7.

The second evaluation item is a result of calculation of a time from the point of the maximum foot speed to the impact point based on the difference between the point of the maximum foot speed and the impact point. That is, the evaluation based on the comparison between the time of the point of the maximum foot speed and the time of the impact point is can be made.

Schematically, the evaluation is good when the point of the maximum speed and the impact point are the same, and the evaluation is poor when the time from the point of the maximum speed to the impact point is long.

For example, the second evaluation item may be based on the information illustrated in FIG. 7.

Here, for example, only any one of the first evaluation item and the second evaluation item may be used, or both may be used.

The third evaluation item is an item indicating whether a knee joint is used well.

In the present example, whether the knee joint is used well by checking the foot speed and the below-knee speed.

For example, the evaluation based on deceleration rate of the impact speed may be performed from the maximum below-knee speed during the kick. As an example, a good deceleration rate may be determined with reference to a kick of a high-level player.

For example, the difference between the maximum foot speed and the maximum below-knee speed during the kick may be calculated, and the evaluation may be performed based on how much the foot speed is increased by swinging down the foot. As an example, the evaluation may be good when the foot speed is greatly increased.

For example, the third evaluation item may be based on the information illustrated in FIGS. 8A, 8B, and 8C.

As a viewpoint of the evaluation as to whether the knee joint is used well, there are the following viewpoints regarding the first posture H1 to the third posture H3.

That is, regarding the first posture H1, there is a viewpoint that the below-knee speed is increased because the foot is swung down with the knee bent.

Further, regarding the second posture H2, there is a viewpoint that the below-knee part is decelerated and the foot speed is increased because the foot is swung down next.

Furthermore, regarding the third posture H3, there is a viewpoint that a good impact is made at a time when the foot speed is increased and the foot speed becomes the highest.

In the good image shown in FIG. 8B, the below-knee speed is maximized, then, the foot speed is greatly increased, and the below-knee part is decelerated over the impact.

On the other hand, in the poor image shown in FIG. 8C, the below-knee speed is maximized, then, the foot speed is not so much increased, and the below-knee part is not so much decelerated over the impact.

The fourth evaluation item is an item indicating whether the foot is firmly raised and whether the foot is firmly swung.

For example, the fourth evaluation item may be based on the information illustrated in FIGS. 9A and 9B.

In the present example, an angle at which the foot is raised is calculated from the below-knee posture. Then, as in the twelfth posture H12, an angle at which the foot is raised most backward is detected, and whether the foot is firmly raised is evaluated based on the angle. Further, as in the fourteenth posture H14, an angle at which the foot is raised most forward is detected, and whether the foot is firmly swung is evaluated based on the angle.

In the present example, schematically, a determination that the evaluation is good when the foot is greatly raised backward and the foot is greatly swung forward.

Here, in the present example, the first evaluation item to the fourth evaluation item are shown, but as another example, only some of the first evaluation item to the fourth evaluation item may be used for the evaluation, and another evaluation item may be used for the evaluation.

In the processing T7 illustrated in FIG. 10, the evaluation report K2 is generated in the server device 12.

In the present example, the report generation unit 135 generates the evaluation report K2 according to the levels of the evaluation results of the first to fourth evaluation items.

The report generation unit 135 may generate the evaluation report K2 using the advice information generated by the advice unit 134.

Here, for example, the advice unit 134 may generate advice information according to the levels of the evaluation results of the first to fourth evaluation items. For example, the generation may be automatically performed, or may be supported by an operation of a coach or the like of the user 51.

As an example, information on advice comments according to the levels of the evaluation results of the first to fourth evaluation items may be prepared in advance as correspondence information of a table format or the like and stored in the storage unit 114. In this case, the advice unit 134 generates advice information according to the levels of the evaluation results of the first to fourth evaluation items based on the correspondence information.

Further, the advice comment can be edited by a coach or the like of the user 51.

For example, the advice unit 134 may have a function of machine learning, and when the advice comment is edited, may learn a correspondence relationship between the edited advice comment and the levels of the evaluation results of the first to fourth evaluation items, and, regarding the next and subsequent measurement data K1, generate the advice information using the information on the learned advice comment based on the learning result according to the levels of the evaluation results of the first to fourth evaluation items.

Here, in the machine learning, for example, information of a table that associates the levels of the evaluation results of the first to fourth evaluation items with any of a plurality of types of fixed advice comments may be learned.

As described above, in the motion evaluation system 1 and the control method thereof according to the present embodiment, the motion of the subject can be evaluated even when it is difficult to wear the sensor on the foot of the subject.

In the motion evaluation system 1 and the control method thereof according to the present embodiment, for example, the movement of the foot can be estimated based on the output from the sensor attached below the knee of the subject and the below-knee length of the subject, and the motion with the estimated movement of the foot can be evaluated.

Further, in the motion evaluation system 1 and the control method thereof according to the present embodiment, for example, it is not necessary to attach a sensor to the ball 61, and thus the motion can be evaluated without a plurality of sensors including a sensor attached to the subject and a sensor attached to the ball. For example, when a sensor attached to the subject and a sensor attached to the ball are used, a large processing load may be applied to securement of time synchronization between these sensors, synthesis of output values from these sensors, and the like.

Here, in the present embodiment, a case where the configuration according to the present embodiment is applied to soccer has been described, but the same configuration as in the present embodiment may be applied to other sports.

As an example, in the present embodiment, a configuration in which the motion of the user 51 kicking the soccer ball 61 has been described, but as another example, a configuration similar to that of the present embodiment may be applied to an evaluation of a motion when kicking a ball of another sport.

Examples of the other sports include rugby.

Configuration examples according to the embodiment are illustrated.

As a configuration example, the control method for the motion evaluation system 1 has the following configuration.

The motion evaluation system 1 includes the sensor attached at a predetermined wearing position below the knee of the subject, and the server device 12 that generates the evaluation report K2 based on the detection result of the sensor.

The control method for the motion evaluation system 1 includes a first step to a fifth step.

In the first step, the sensor is caused to detect motion information indicating the motion below the knee of the subject.

In the second step, the sensor is caused to transmit the motion information to the server device 12.

In the third step, the server device 12 is caused to acquire the length information on the length from the below-knee part to the foot of the subject.

In the fourth step, the server device 12 is caused to estimate the movement of the foot of the subject from the received motion information and the acquired length information.

In the fifth step, the server device 12 is caused to perform an evaluation on the estimated movement of the foot.

Therefore, in the control method for the motion evaluation system 1, for example, even when it is difficult to wear the sensor on the foot of the subject, the motion of the subject can be evaluated.

Here, in the present embodiment, the user 51 is an example of the subject.

In the present embodiment, the sensor provided in the measurement device 11 is an example of the sensor. The number of sensors may be one or more.

Further, the predetermined wearing position may be various positions, and may be, for example, the below-knee position illustrated in FIG. 3A, the first wearing position E1 illustrated in FIG. 3C, the second wearing position E2 illustrated in FIG. 3D, or the like.

In the present embodiment, as an example, the motion information indicating the motion at a predetermined wearing position below the knee of the subject may be used as the motion information indicating the motion below the knee of the subject. In this case, the motion information indicating the motion at the predetermined wearing position may be, for example, information contained in the measurement data K1 when the user 51 kicks the ball 61, or information obtained by calculation or the like based on the information. Here, the measurement data K1 is, for example, data measured for the predetermined wearing position of the sensor.

As another example, motion information indicating a motion at a position below the knee of the subject and other than the predetermined wearing position may be used as the motion information indicating the motion below the knee of the subject. In this case, the motion information indicating the motion at the position other than the predetermined wearing position may be obtained by, for example, calculation based on the measurement data K1.

In the present embodiment, the information on the below-knee length of the user 51 is an example of the length information on the length from the below-knee part to the foot of the subject.

In the present embodiment, the movement of the foot when the user 51 kicks the ball 61 is an example of the movement of the foot of the subject.

As a configuration example, the control method for the motion evaluation system 1 has the following configuration.

In the fifth step, the server device 12 specifies a feature point of the motion information from the motion information detected by the sensor, and extracts the feature point as evaluation data of the motion of the foot.

Therefore, in the control method for the motion evaluation system 1, for example, the evaluation section can be automatically extracted from the data detected by the sensor.

Here, in the present embodiment, each point at the first time t1 to the fourth time t4 shown in FIG. 6 is an example of the feature point of the motion information.

As a configuration example, the control method for the motion evaluation system 1 includes a sixth step.

In the sixth step, the server device 12 is caused to output advice according to the evaluation.

Therefore, in the control method for the motion evaluation system 1, for example, advice can be automatically generated.

As a configuration example, the control method for the motion evaluation system 1 has the following configuration.

In the third step, the server device 12 is caused to receive information on the height of the subject.

In the third step, the server device 12 is caused to acquire length information based on the height of the subject.

Therefore, in the control method for the motion evaluation system 1, for example, the length from the below-knee part to the foot of the subject is acquired based on the height of the subject.

As a configuration example, the control method for the motion evaluation system 1 has the following configuration.

In the fifth step, the server device 12 is caused to estimate the motion information on the foot rotated around the knee of the subject and perform an evaluation on the speed of the foot.

Therefore, in the control method for the motion evaluation system 1, for example, the speed of the kick can be evaluated from the data below the knee.

As a configuration example, the control method for the motion evaluation system 1 has the following configuration.

In the fifth step, the server device 12 is caused to specify a specific time from the motion information on the foot and perform an evaluation on the kick force of the foot at the specific time.

Therefore, in the control method for the motion evaluation system 1, for example, as in the first evaluation item or the second evaluation item, the kick force can be evaluated from the detection value in the sensor.

Here, the specific time may be, for example, a time of the impact point.

As a configuration example, the control method for the motion evaluation system 1 has the following configuration.

In the fifth step, the server device 12 is caused to perform an evaluation by comparing the maximum speed from the motion information on the foot with the foot speed at the specific time.

Therefore, in the control method for the motion evaluation system 1, for example, as in the first evaluation item or the second evaluation item, the time of the kick can be evaluated from the detection value in the sensor.

As a configuration example, the control method for the motion evaluation system 1 has the following configuration.

In the fifth step, the server device 12 is caused to perform an evaluation by comparing the speed below the knee, which is the actual measurement value detected by the sensor, with the foot speed, which is the estimated value.

Therefore, in the control method for the motion evaluation system 1, for example, as in the third evaluation item, whether the knee joint is used well can be evaluated from the detection value in the sensor.

Here, as the speed below the knee, for example, a speed at a predetermined wearing position below the knee may be used, or a speed at a position other than the predetermined wearing position below the knee may be used.

As a configuration example, the control method for the motion evaluation system 1 has the following configuration.

In the fifth step, the server device 12 is caused to evaluate a swing-up angle of the foot according to the posture below the knee after the specific time.

Therefore, in the control method for the motion evaluation system 1, for example, as in the fourth evaluation item, the swing-up of the foot can be evaluated from the detection value in the sensor.

Here, as the posture below the knee, for example, a posture at a predetermined wearing position below the knee may be used, or a posture at a position other than the predetermined wearing position below the knee may be used.

As a configuration example, the control method for the motion evaluation system 1 has the following configuration.

The control method for the motion evaluation system 1 uses the sensor worn at the predetermined wearing position below the knee of the subject and the server device 12 to generate the evaluation report K2 based on the detection result of the sensor.

The control method for the motion evaluation system 1 includes detecting the motion information indicating the motion below the knee of the subject, acquiring the length information on the length from the below-knee part to the foot of the subject, estimating the movement of the foot of the subject from the detected motion information and the acquired length: information, and performing an evaluation on the estimated movement of the foot.

Therefore, in the control method for the motion evaluation system 1, for example, even when it is difficult to wear the sensor on the foot of the subject, the motion of the subject can be evaluated.

As a configuration example, a control method for a computer has the following configuration.

The control method for the computer includes the following steps.

The control method for the computer includes a step of causing the computer to acquire the motion information indicating the motion of the subject below the knee from the sensor attached at the predetermined wearing position below the knee of the subject.

The control method for the computer includes a step of causing the computer to acquire the length information on the length from the below-knee part to the foot of the subject.

The control method for the computer includes a step of causing the computer to estimate the movement of the foot of the subject from the acquired motion information and the acquired length information.

The control method for the computer includes a step of causing the computer to perform an evaluation on the estimated movement of the foot.

Therefore, in the control method for the computer, for example, even when it is difficult to wear the sensor on the foot of the subject, the motion of the subject can be evaluated.

Here, the computer may be, for example, a computer forming the server device 12.

As a configuration example, a program has the following configuration.

The program is a program for causing a computer to execute each of the steps described above.

Therefore, in the program, for example, even when it is difficult to wear the sensor on the foot of the subject, the motion of the subject can be evaluated.

As a configuration example, the motion evaluation system 1 has the following configuration.

The motion evaluation system 1 includes the sensor attached at the predetermined wearing position below the knee of the subject and detects the motion information indicating the motion below the knee of the subject, receives the motion information from the sensor, estimates the motion of the foot of the subject from the received motion information and the length information on the length from the below-knee part to the foot of the subject, and evaluates the estimated motion of the foot.

Therefore, in the motion evaluation system 1, for example, even when it is difficult to wear the sensor on the foot of the subject, the motion of the subject can be evaluated.

A program for implementing the function of any configuration in any device described above may be recorded in a computer-readable storage medium and the program may be read and executed by a computer system. The “computer system” referred to here includes an operating system or hardware such as peripheral equipment. The “computer-readable storage medium” is a portable medium such as a flexible disc, a magneto-optical disc, a read only memory (ROM), or a compact disc (CD)-ROM or a storage device such as a hard disk incorporated in the computer system. The “computer-readable storage medium” includes a storage medium that retains a program for a certain period of time like a volatile memory on the inside of the computer system serving as a server or a client when the program is transmitted via a network such as the Internet or a communication line such as a telephone line. The volatile memory may be a RAM. The storage medium may be a non-transitory storage medium.

The program described above may be transmitted from a computer system in which the program is stored in a storage device or the like to another computer system via a transmission medium or by a transmission wave in the transmission medium. The “transmission medium” for transmitting the program refers to a medium having a function of transmitting information like a network such as the Internet or a communication line such as a telephone line.

The program described above may be a program for implementing some of the functions described above. The program described above may be a program that can implement the functions described above in combination with a program already recorded on the computer system, a so-called differential file. The differential file may be called a differential program.

The function of any configuration in any device described above may be implemented by a processor. Each processing in the embodiment may be implemented by a processor that operates based on information such as a program and a computer-readable storage medium that stores the information such as the program. In the processor, the functions of the units may be implemented by individual hardware or the functions of the units may be implemented by integrated hardware. The processor may include hardware and the hardware may include at least one of a circuit that processes a digital signal and a circuit that processes an analog signal. The processor may be configured using one or both of one or more of circuit devices implemented on a circuit board and one or more of circuit elements. An integrated circuit (IC) or the like may be used as the circuit device and a resistor, a capacitor, or the like may be used as the circuit element.

The processor may be a CPU. However, the processor is not limited to the CPU, but various processors such as a GPU (Graphics Processing Unit) or a DSP (Digital Signal Processor) may be used. The processor may be a hardware circuit by an ASIC (Application Specific Integrated Circuit). The processor may include a plurality of CPUs or may include a hardware circuit by a plurality of ASICS. The processor may include a combination of a plurality of CPUs and a hardware circuit by a plurality of ASICS. The processor may include one or more of an amplifier circuit, a filter circuit, and the like that process an analog signal.

While the embodiment is described in detail above with reference to the drawings, a specific configuration is not limited to the embodiment and include design and the like in a range not departing from the gist of the present disclosure.

### Appendices

Configuration Examples 1 to 13 will be described below.

A lower-order configuration example may or may not be applied to a higher-order configuration example.

A lower-order configuration example applicable to any one of two or more higher-order configuration examples may be applied to any configuration example among the two or more higher-order configuration examples and, when there are two or more application examples in this way, a configuration example lower in order than the lower-order configuration example may be applied to any application example among the two or more application examples.

### Configuration Example 1

A control method of a motion evaluation system including a sensor attached at a predetermined wearing position below a knee of a subject, and a server device that generates an evaluation report based on a detection result of the sensor, the method includes a first step of causing the sensor to detect motion information indicating a motion below the knee of the subject, a second step of causing the sensor to transmit the motion information to the server device, a third step of causing the server device to acquire length information based on a height of the subject, a fourth step of causing the server device to estimate a movement of a foot of the subject from the received motion information and the acquired length information, and a fifth step of causing the server device to perform an evaluation on the estimated movement of the foot.

### Configuration Example 2

In the control method of the motion evaluation system according to Configuration Example 1, in the fifth step, the server device is caused to specify a feature point of the motion information from the motion information detected by the sensor and extract the feature point as evaluation data of a motion of the foot.

### Configuration Example 3

The control method of the motion evaluation system according to Configuration Example 1 or Configuration Example 2, further includes a sixth step of causing the server device to output advice according to the evaluation.

### Configuration Example 4

In the control method of the motion evaluation system according to any one of Configuration Example 1 to Configuration Example 3, in the third step, the server device is caused to acquire the length information from a below-knee part to the foot of the subject based on the height of the subject.

### Configuration Example 5

In the control method of the motion evaluation system according to any one of Configuration Example 1 to Configuration Example 4, in the fifth step, the server device is caused to estimate the motion information on the foot rotated around the knee of the subject and perform an evaluation on a speed of the foot.

### Configuration Example 6

In the control method of the motion evaluation system according to any one of Configuration Example 1 to Configuration Example 5, in the fifth step, the server device is caused to specify a specific time from the motion information on the foot and perform an evaluation on a kick force of the foot at the specific time.

### Configuration Example 7

In the control method of the motion evaluation system according to any one of Configuration Example 1 to Configuration Example 6, in the fifth step, the server device is caused to perform the evaluation by comparing a maximum speed of the foot from the motion information with a speed of the foot at a specific time.

### Configuration Example 8

In the control method of the motion evaluation system according to any one of Configuration Example 1 to Configuration Example 7, in the fifth step, the server device is caused to perform an evaluation by comparing a speed below the knee, which is an actual measurement value detected by the sensor, with a speed of the foot, which is an estimated value.

### Configuration Example 9

In the control method of the motion evaluation system according to any one of Configuration Example 1 to Configuration Example 8, in the fifth step, the server device is caused to evaluate a swing-up angle of the foot according to a posture below the knee after a specific time.

### Configuration Example 10

A control method for a motion evaluation system that uses a sensor attached at a predetermined wearing position below a knee of a subject and a server device to generate an evaluation report based on a detection result of the sensor, the method includes detecting motion information indicating a motion below the knee of the subject, acquiring length information based on a height of the subject, estimating a movement of a foot of the subject from the detected motion information and the acquired length information, and performing an evaluation on the estimated movement of the foot.

### Configuration Example 11

A control method for a computer includes causing the computer to acquire motion information indicating a motion of below a knee of a subject from a sensor attached at a predetermined wearing position below the knee of the subject, causing the computer to acquire length information based on a height of the subject, causing the computer to estimate a movement of a foot of the subject from the acquired motion information and the acquired length information, and causing the computer to perform an evaluation on the estimated movement of the foot.

### Configuration Example 12

A non-transitory computer-readable storage medium storing a program for causing the computer to execute the steps according to Configuration Example 11.

### Configuration Example 13

A motion evaluation system includes a sensor attached at a predetermined wearing position below a knee of a subject and detecting motion information indicating a motion below the knee of the subject, and a server device receiving the motion information from the sensor, estimating a movement of a foot of the subject from the received motion information and length information based on a height of the subject, and performing an evaluation on the estimated movement of the foot.

What is claimed is:

1. A control method for a motion evaluation system including a sensor attached at a predetermined wearing position below a knee of a subject, and a server device that generates an evaluation report based on a detection result of the sensor, the method comprising:
a first step of causing the sensor to detect motion information indicating a motion below the knee of the subject;
a second step of causing the sensor to transmit the motion information to the server device;
a third step of causing the server device to acquire length information based on a height of the subject;
a fourth step of causing the server device to estimate a movement of a foot of the subject from the received motion information and the acquired length information; and
a fifth step of causing the server device to perform an evaluation on the estimated movement of the foot.

2. The control method for a motion evaluation system according to claim 1, wherein
in the fifth step, the server device is caused to specify a feature point of the motion information from the motion information detected by the sensor and extract the feature point as evaluation data of a motion of the foot.

3. The control method for a motion evaluation system according to claim 1, further comprising a sixth step of causing the server device to output advice according to the evaluation.

4. The control method for a motion evaluation system according to claim 1, wherein
in the third step, the server device is caused to acquire the length information from a below-knee part to the foot of the subject based on the height of the subject.

5. The control method for a motion evaluation system according to claim 1, wherein
in the fifth step, the server device is caused to estimate the motion information on the foot rotated around the knee of the subject and perform an evaluation on a speed of the foot.

6. The control method for a motion evaluation system according to claim 1, wherein
in the fifth step, the server device is caused to specify a specific time from the motion information on the foot and perform an evaluation of a kick force of the foot at the specific time.

7. The control method for a motion evaluation system according to claim 1, wherein
in the fifth step, the server device is caused to perform the evaluation by comparing a maximum speed of the foot from the motion information with a speed of the foot at a specific time.

8. The control method for a motion evaluation system according to claim 1, wherein
in the fifth step, the server device is caused to perform an evaluation by comparing a speed below the knee, which is an actual measurement value detected by the sensor, with a speed of the foot, which is an estimated value.

9. The control method for a motion evaluation system according to claim 1, wherein
in the fifth step, the server device is caused to evaluate a swing-up angle of the foot according to a posture below the knee after a specific time.

10. A control method for a computer comprising:
causing the computer to acquire motion information indicating a motion below a knee of a subject from a sensor attached at a predetermined wearing position below the knee of the subject;
causing the computer to acquire length information based on a height of the subject;
causing the computer to estimate a movement of a foot of the subject from the acquired motion information and the acquired length information; and
causing the computer to perform an evaluation on the estimated movement of the foot.

11. A non-transitory computer-readable storage medium storing a program for causing the computer to execute the respective steps according to claim 10.

12. A motion evaluation system comprising:
a sensor attached at a predetermined wearing position below a knee of a subject and detecting motion information indicating a motion below the knee of the subject; and
a server device receiving the motion information from the sensor, estimating a movement of a foot of the subject from the received motion information and length information based on a height of the subject, and performing an evaluation on the estimated movement of the foot.
